(19) United States
(12) Patent Application Publication
(10) Pub. No.: US 2023/0265525 A1
(43) Pub. Date: Aug. 24, 2023

(54) METHODS FOR DETECTING HOMOLOGOUS RECOMBINATION DEFICIENCY IN CANCER PATIENTS

(71) Applicant: GeneGenieDx Corp, San Jose, CA (US)

(72) Inventors: Minzhe Zhang, Dallas, TX (US); Wenying Pan, Cupertino, CA (US); Qing Zhang, Chandler, AZ (US); Xiao Yang, Dublin, CA (US)

(21) Appl. No.: 18/172,322

(22) Filed: Feb. 22, 2023

Related U.S. Application Data
(60) Provisional application No. 63/312,823, filed on Feb. 22, 2022.

(51) Int. Cl. C12Q 1/6886

(52) U.S. Cl. CPC C12Q 1/6886; C12Q 2600/106; C12Q 2600/156

(57) ABSTRACT

The present disclosure provides methods and compositions, e.g., kits, for detecting homologous recombination deficiency in a cancer patient. In certain embodiments, the methods disclosed are based on genomic copy number summarization and deep learning model to detect homologous recombination deficiency from extremely low coverage whole genome sequencing data.

### CROSS-REFERENCE TO RELATED APPLICATIONS

This application claims the priority of U.S. provisional application No. 63/312,823, filed Feb. 22, 2022, the entire disclosure of which is incorporated herein by reference.

### FIELD OF THE INVENTION

The present invention generally relates to cancer diagnosis, prognosis and treatment. In particular, the present invention relates the methods of detecting deficiency in the DNA homologous recombination pathways in a cancer patient.

### BACKGROUND

Homologous recombination is a highly accurate DNA repair mechanism in eukaryotic cells. Deficiency in the homologous recombination pathway, i.e., homologous recombination deficiency (HRD) occurs when the genes involved in the homologous recombination pathway, such as BRCA1, BRCA2, are inactivated. HRD results in a disorder in the repair of DNA double-stranded breaks, leading to tumorigenesis (see Lord, C. J., & Ashworth, A. (2012) The DNA damage response and cancer therapy. Nature, 481(7381), 287-294; Venkitaraman, A. R. (2014) Cancer suppression by the chromosome custodians, BRCA1 and BRCA2. Science, 343(6178), 1470-1475). HRD is frequently observed in breast and ovarian cancer patients and is shown to be associated with patient response to poly(ADP-ribose) polymerase (PARP) inhibitors like Veliparib (Nik-Zainal, S. et al. (2016) Landscape of somatic mutations in 560 breast cancer whole-genome sequences. Nature, 534(7605), 47-54; Coleman, R. L. et al. (2019) Veliparib with first-line chemotherapy and as maintenance therapy in ovarian cancer. New England Journal of Medicine, 381(25), 2403-2415). Therefore, identification of HRD is a good marker to select patients that are more likely to benefit from PARP inhibitor treatment.

Currently, there are two FDA-approved HRD companion diagnostic tests, myChoice CDx by Myriad Genetics and FoundationOne CDx by Foundation Medicine, and several other gene panel tests covering BRCA1 and BRCA2 are available on the market. Both myChoice CDx and FoundationOne CDx use the deep sequencing approach to identify either the mutation status of BRCA1 and BRCA2 or high genomic instability score, such as loss of heterozygosity (LOH) as positive HRD markers. These approaches typically require high sequencing depth (FoundationOne CDx has 500 median coverage), good tumor fraction (FoundationOne CDx recommend higher than 25%) of FFPE samples and matched normal tissue samples to have good sensitivity. There is a continuing need to develop new methods for detecting HDR in cancer patients.

### SUMMARY OF INVENTION

The present disclosure in one aspect provides a method for detecting deficiency in the DNA homologous recombination pathway in a patient having cancer. In some embodiments, the method comprises: obtaining a whole genome sequencing (WGS) data of a tumor sample from the patient; generating from the WGS data a genome copy number profile; generating from the genome copy number profile a chromosome instability (CI) scores for each chromosome using a deep learning model, thus generating a set of CI scores; generating from the set of CI scores a risk score of deficiency in the DNA homologous recombination pathway; determining that the risk score is greater than a threshold number; and identified the patient as having a risk of deficiency in the DNA homologous recombination pathway. In some embodiment, the method does not require a WGS data of a normal tissue/sample from the patient.

In some embodiments, the threshold number of the risk score is 0.4. In some embodiments, the risk of deficiency in the DNA homologous recombination pathway is high when the risk score is greater than 0.6. In some embodiments, the risk of deficiency in the DNA homologous recombination pathway is moderate when the risk score is greater than 0.4 and less than or equal to 0.6.

In some embodiments, the deep learning model comprises a layer of long short-term memory and a fully connected network.

In some embodiments, the risk score is an average of the set of CI scores weighted by base length of each chromosome, position of each chromosome or position of each CNA break.

In some embodiments, the method for detecting deficiency in the DNA homologous recombination pathway in a cancer patient comprises: obtaining a whole genome sequencing (WGS) data of a tumor sample from the patient, wherein the WGS data has a sequencing depth of 0.05 to 1; generating from the WGS data a genomic alteration profile including copy number alteration, gene fusion, inversion profile or translocation; generating from the genomic alteration profile a number, per genome, of large-scale genomic breakpoints (LGBs), wherein the LGB is a breakpoint between two adjacent genomic segments of different genomic copy number, each such genomic segment being at least a certain threshold number of length long (e.g. 10 megabases); determining that the number of LGBs is greater than a threshold number of LGBs; and identified the patient as having a risk of deficiency in the DNA homologous recombination pathway.

In some embodiments, the threshold number of LGBs is 25. In some embodiments, the patient has a high risk of deficiency in the DNA homologous recombination pathway when the number of LGBs is greater than 35. In some embodiments, the patient has a moderate risk of deficiency in the DNA homologous recombination pathway when the number of LGBs is greater than 25 and less than or equal to 35.

In some embodiments, tumor sample is an FFPE sample or a CTC sample.

In some embodiments, the cancer is selected from breast cancer, ovary cancer, pancreas cancer, head and neck carcinoma and melanoma. In some embodiments, the cancer is breast cancer.

In some embodiments, the genome alteration profile comprises a parameter selected from genomic segments along the genome, copy number of the genomic segments, copy number alteration (CNA) breaks, number of CNA breaks, and a combination thereof. In some embodiments, the genome alteration profile also comprises the number of fusions, inversion and translocation events involved.

In some embodiments, the genome copy number profile is generated by: aligning the WGS data to a reference genome, thereby generating a group of aligned WGS reads along genome, counting the number of aligned WGS reads along the genome, and generating genomic segments along the genome, wherein two adjacent genomic segments have significantly different number of read counts. The fusion or translocation event is identified by identifying the split reads or read pairs that are mapped to different locations within or across chromosomes; the inversion is identified by read pairs that have wrong orientations on the same chromosome.

In some embodiments, the method disclosed herein further comprises administering to the patient a therapeutically effective amount of a PARP inhibitor and/or an alkylating agent. In some embodiments, the PARP inhibitor and/or alkylating agent is selected from iniparib, Olaparib, rucaparib, CEP9722, MK4827, BMN673, 3-aminobenzaide, platinum complexes, chlormethine, chlorambucil, melphalan, cyclophosphamide, ifosfamide, estramustine, carmustine, lomustine, fotemustine, streptozocin, busulfan, pipobroman, procarbazine, dacarbazine, thiotepa and temozolomide.

In another aspect, the present disclosure provides a method for treating cancer in a patient. In some embodiments, the method comprises administering to the patient a therapeutically effective amount of a PARP inhibitor and/or an alkylating agent, wherein the patient has been identified as having a risk of deficiency in the DNA homologous recombination pathway by the method disclosed herein.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows the schematic of a deep learning model for generating CI scores.

FIG. 2 shows the LGB scores in HRD and non-HRD patients.

FIG. 3 shows the correlation between LGB and HRD risk scores of 0.5 coverage and other coverage.

FIG. 4 shows the correlation between LGB and HRD risk scores of original samples and diluted samples.

FIG. 5 shows the results of LGB test in exemplary samples with sequencing depth from 0.01 to 0.5.

FIG. 6 shows the correlation between the LGB scores at 0.5 coverage and the LGB scores at 0.2 or 0.1 coverages.

FIG. 7 shows the results of HRD risk test in exemplary samples with sequencing depth from 0.01 to 0.5.

FIG. 8 shows the correlation between the HRD risk scores at 0.5 coverage and the HRD risk scores at 0.2, 0.1 or 0.05 coverages.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### Definitions

The following definitions are provided to assist the reader. Unless otherwise defined, all terms of art, notations and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not necessarily be construed to represent a substantial difference over the definition of the term as generally understood in the art.

As used herein, the singular forms “a”, “an” and “the” include plural references unless the context clearly dictates otherwise.

As used herein, the term “administering” means providing a pharmaceutical agent or composition to a subject, and includes, but is not limited to, administering by a medical professional and self-administering.

As used herein, the term “cancer” refers to any diseases involving an abnormal cell growth and include all stages and all forms of the disease that affects any tissue, organ or cell in the body. The term includes all known cancers and neoplastic conditions, whether characterized as malignant, benign, soft tissue, or solid, and cancers of all stages and grades including pre- and post-metastatic cancers. In general, cancers can be categorized according to the tissue or organ from which the cancer is located or originated and morphology of cancerous tissues and cells. As used herein, cancer types include, without limitation, acute lymphoblastic leukemia (ALL), acute myeloid leukemia, adrenocortical carcinoma, anal cancer, astrocytoma, childhood cerebellar or cerebral, basal-cell carcinoma, bile duct cancer, bladder cancer, bone tumor, brain cancer, cerebellar astrocytoma, cerebral astrocytoma/malignant glioma, ependymoma, medulloblastoma, supratentorial primitive neuroectodermal tumors, visual pathway and hypothalamic glioma, breast cancer, Burkitt’s lymphoma, cervical cancer, chronic lymphocytic leukemia, chronic myelogenous leukemia, colon cancer, emphysema, endometrial cancer, ependymoma, esophageal cancer, Ewing’s sarcoma, retinoblastoma, gastric (stomach) cancer, glioma, head and neck cancer, heart cancer, Hodgkin lymphoma, islet cell carcinoma (endocrine pancreas), Kaposi sarcoma, kidney cancer (renal cell cancer), laryngeal cancer, leukemia, liver cancer, lung cancer, neuroblastoma, non-Hodgkin lymphoma, ovarian cancer, pancreatic cancer, pharyngeal cancer, prostate cancer, rectal cancer, renal cell carcinoma (kidney cancer), retinoblastoma, Ewing family of tumors, skin cancer, stomach cancer, testicular cancer, throat cancer, thyroid cancer, vaginal cancer.

It is noted that in this disclosure, terms such as “comprises”, “comprised”, “comprising”, “contains”, “containing” and the like have the meaning attributed in United States Patent law; they are inclusive or open-ended and do not exclude additional, un-recited elements or method steps. Terms such as “consisting essentially of” and “consists essentially of” have the meaning attributed in United States Patent law; they allow for the inclusion of additional ingredients or steps that do not materially affect the basic and novel characteristics of the claimed invention. The terms “consists of” and “consisting of” have the meaning ascribed to them in United States Patent law; namely that these terms are close ended.

The terms “determining,” “assessing,” “assaying,” “measuring” and “detecting” can be used interchangeably and refer to both quantitative and semi-quantitative determinations. Where either a quantitative and semi-quantitative determination is intended, the phrase “determining a level” of a polynucleotide or polypeptide of interest or “detecting” a polynucleotide or polypeptide of interest can be used.

The term “nucleic acid” and “polynucleotide” are used interchangeably and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, shRNA, single-stranded short or long RNAs, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “subject” refers to a human or any non-human animal (e.g., mouse, rat, rabbit, dog, cat, cattle, swine, sheep, horse or primate). A human includes pre and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “therapeutically effective amount” means the amount of agent that is sufficient to prevent, treat, reduce and/or ameliorate the symptoms and/or underlying causes of any disorder or disease, or the amount of an agent sufficient to produce a desired effect on a cell. In one embodiment, a “therapeutically effective amount” is an amount sufficient to reduce or eliminate a symptom of a disease. In another embodiment, a therapeutically effective amount is an amount sufficient to overcome the disease itself.

The term “treatment,” “treat,” or “treating” refers to a method of reducing the effects of a cancer (e.g., breast cancer, lung cancer, ovarian cancer or the like) or symptom of cancer. Thus, in the disclosed method, treatment can refer to a 10%, 20%, 30%, 40%, 50%, 60%, 70%), 80%), 90%), or 100% reduction in the severity of a cancer or symptom of the cancer. For example, a method of treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100% or any percent reduction between 10 and 100% as compared to native or control levels. It is understood that treatment does not necessarily refer to a cure or complete ablation of the disease, condition, or symptoms of the disease or condition.

### Cancer and Homologous Recombination Deficiency

The development of cancer is a multifactorial process and comprises key events such as the ability of unlimited replication, the induction of angiogenesis and the activation of invasion and metastasis. One major underlying mechanism of cancer development is the emergence of genomic instability caused by genetic mutations arising from exogenously or endogenously caused DNA damage or failures in DNA damage repair. Organisms have evolved different strategies to cope with various forms of DNA damage. Incorrect bases incorporated during replication are removed by proteins of the mismatch repair (MMR) pathway. The nucleotide excision repair (NER) mechanism identifies structural distortions within the DNA double-strand and removes the affected bases. The base excision repair (BER) pathway is activated by damaged DNA bases. In response to double-strand breaks, two major repair pathways are available: the exact mechanism of homologous recombination (HR) and the error-prone non-homologous end joining (NHEJ).

DNA double-strand breaks (DSBs) can be caused by exogenous sources such as ionizing radiation or endogenous sources such as byproducts of cell metabolism. DSBs caused by internal factors, such as replication block, can usually be directly repaired by using the sister chromatid, which is located in close proximity, as a template for homologous recombination. On the other hand, DSBs caused by ionizing radiation mainly occurs at the densely packed chromatin structure. Therefore, an interaction with intact homologous sequences as a template for HR is not possible. In order to deal with such types of DSBs in condensed chromatin structures, vertebrates frequently use NHEJ to simply re-ligate the DSB end strands. Another likely reason for NHEJ being the predominant DSB repair mechanism in humans might because HR preferably takes place in the S/G2 phase while NHEJ is dominant in the G1 phase, which is longer than S/G2.

Homologous recombination is an accurate repair mechanism to cope with DSBs because it uses an intact copy of the DNA from the sister chromatid or the homologous chromosome as a matrix to repair the break. In a first step, the damage is recognized by the MRE11-RAD50-NBS1 (MRN) complex, which activates ATM (Ataxia telangiectasia mutated) kinase. Upon DNA 5′-end resection, the replication protein (RPA) coats the single-strand DNA regions and activates ATR (Ataxia telangiectasia and Rad3-related) kinase. RPA is then replaced by RAD51 with the help of further repair-associated proteins, such as CHEK2, BRCA1, BRCA2 and PALB2, which are loaded with RAD51. Subsequently, the defective DNA strand attaches to its sister chromatid, which is used as a template for DNA resynthesis.

The HR pathway includes proteins that are important for the repair of double-strand DNA breaks, such as BRCA1, BRCA2, PALB2/FANCN, BRIP1/FANCJ, BARD1, RAD51 and RAD51 paralogs (RAD51B, RAD51C, RAD51D, XRCC2, XRCC3). When the gene for any of these proteins is mutated or under-expressed, errors in DNA repair may occur and can eventually cause cancer. Although not yet found recurrently mutated in human tumors, other actors of the HR pathway may potentially be deregulated in cancers, such as FANCA, FANCB, FANCC, FANCD2, FANCE, FANCG, FANCI, FANCL, FANCM, FAN1, SLX4/FANCP or ERCC1. As used herein, deficiency in the HR pathway and HR deficiency are used interchangeably and refer to a condition in which one or several of the proteins involved in the HR pathway for repairing DNA is deficient or inactivated.

The core component of non-homologous end joining (NHEJ) pathway is the Ku70-Ku80 complex, which is able to bind ends of double-strand broken DNA and recruit DNA-PKcs to initiate NHEJ. Further factors process the broken DNA to build DNA ends compatible for ligation. Subsequently, DNA ligase IV (LIG4) is simultaneously recruited with XRCC4 and an XRCC4-like factor (XLF) to ligate the processed DNA and restore genome integrity. During NHEJ, changes within the DNA sequence or ligation of random blunt ends may take place. Thus, chromosomal integrity is prone to get lost, giving rise to chromosomal rearrangements.

Cancer patients having HRD have been treated by poly (ADP-ribose) polymerase (PARP) inhibitors, which disable single-strand break repair. When homologous recombination deficiency (HRD) occurs in a cell, the cell is forced to switch to the error prone NHEJ pathway to repair DSBs. This leads to the accumulation of genomic damages and fosters genomic instability. When HRD and PARP inhibition occur simultaneously in a cell, the DNA damage repair mechanism is greatly impaired, leading to synthetic lethality and forced cell death.

Nevertheless, it remains challenging to define those patients having HRD who might benefit from PARPi therapy. Alternations of genes encoding proteins in the HR pathway, such as BRCA1 and BRCA2, are commonly used to identify patients with HRD. However, in a recent clinical trial in ovarian cancer, it was shown that nearly 20% of the study population was HRD positive without having a BRCA mutation (Miller, R.E. et al. (2020) ESMO recommendations on predictive biomarker testing for homologous recombination deficiency and PARP inhibitor benefit in ovarian cancer. Ann. Oncol. Off. J. Eur. Soc. Med. Oncol. 31, 1606-1622). Therefore, there is a need to identify patients having deficiency in the HR pathway.

### Methods of Detecting Homologous Recombination Deficiency

The present disclosure in one aspect provides methods of detecting deficiency in the DNA homologous recombination pathway in a patient having cancer. In some embodiments, the method involves using whole genome sequencing (WGS) data generated from a tumor sample of the patient to detect chromosome instability in the sample.

The tumor sample used in the methods disclosed herein refers a biological sample or a sample from a biological source that contains one or more tumor cells. Biological samples include samples from body fluids, e.g., blood, plasma, serum, or urine, or samples derived, e.g., by biopsy, from cells, tissues or organs, preferably tumor tissue suspected to include or essentially consist of cancer cells. The tumor sample can be fresh tumor sample or frozen tumor sample. The tumor sample can be obtained by biopsy, e.g., through surgery or needle biopsy.

In some embodiments, the tumor samples used in the methods disclosed herein have low tumor proportion or low tumor DNA proportion. Such kind of tumor samples include formalin-fixed paraffin-embedded (FFPE) tissue samples, single cell sample, and circulating tumor cells. In such case, WGS data generated from the tumor sample and used in the methods disclosed herein have very low sequencing depth. As used herein, the sequencing depth refers to the ratio of the total number of bases obtained by sequencing to the size of the genome or the average number of times each base is measured in the genome. In some embodiments, the WGS data used in the methods disclosed herein have a depth of 0.05 to 0.5, e.g., 0.05, 0.1, 0.15, 0.2, 0.25, 0.3, 0.35, 0.4, 0.45, and 0.5.

In some embodiments, WGS is performed using DNA isolated from the tumor sample. DNA can be isolated from the tumor sample using a variety of methods. Standard methods for DNA extraction from tissue or cells are described in, for example, Ausubel et al., Current Protocols of Molecular Biology (1997) John Wiley & Sons, and Sambrook and Russell, Molecular Cloning: A Laboratory Manual 3rd ed. (2001). Commercially available kits, e.g., QIAamp® DNA Stool Mini Kit (Qiagen) can also be used to isolate DNA from a tumor sample.

### Whole Genome Sequencing Methods

In the present disclosure, the whole genome sequencing data can be generated by any methods known in the art. In some embodiments, the WGS is performed by high throughput sequencing.

High throughput sequencing, or next generation sequencing, by using methods distinguished from traditional methods, such as Sanger sequencing, is highly scalable and able to sequence the entire genome or transcriptome at once. Based on its mechanism, high throughput sequencing includes sequencing-by-synthesis, pyrosequencing, sequencing-by-ligation, and nanopore sequencing.

Sequence-by-synthesis involves synthesizing a complementary strand of the target nucleic acid by incorporating labeled nucleotide or nucleotide analog in a polymerase amplification. Immediately after or upon successful incorporation of a label nucleotide, a signal of the label is measured and the identity of the nucleotide is recorded. The detectable label on the incorporated nucleotide is removed before the incorporation, detection and identification steps are repeated. Examples of sequence-by-synthesis methods are known in the art, and are described for example in U.S. Pat. No. 7,056,676, U.S. Pat. No. 8,802,368 and U.S. Pat. No. 7,169,560, the contents of which are incorporated herein by reference. Sequencing-by-synthesis may be performed on a solid surface (or a microarray or a chip) using fold-back PCR and anchored primers. Target nucleic acid fragments can be attached to the solid surface by hybridizing to the anchored primers, and bridge amplified. This technology is used, for example, in the Illumina® sequencing platform.

Pyrosequencing involves hybridizing the target nucleic acid regions to a primer and extending the new strand by sequentially incorporating deoxynucleotide triphosphates corresponding to the bases A, C, G, and T (U) in the presence of a polymerase. Each base incorporation is accompanied by release of pyrophosphate, converted to ATP by sulfurylase, which drives synthesis of oxyluciferin and the release of visible light. Since pyrophosphate release is equimolar with the number of incorporated bases, the light given off is proportional to the number of nucleotides adding in any one step. The process is repeated until the entire sequence is determined.

Sequencing-by-ligation is a DNA sequencing method that uses the enzyme DNA ligase to identify the nucleotide present at a given position in a DNA sequence. Unlike most currently popular DNA sequencing methods, sequencing-by-ligation does not use a DNA polymerase to create a second strand. Instead, the mismatch sensitivity of a DNA ligase enzyme is used to determine the underlying sequence of the target DNA molecule. Sequencing-by-ligation relies upon the sensitivity of DNA ligase for base-pairing mismatches. The target molecule to be sequenced is a single strand of unknown DNA sequence, flanked on at least one end by a known sequence. A short “anchor” strand is brought in to bind the known sequence. A mixed pool of probe oligonucleotides is then brought in (eight or nine bases long), labeled (typically with fluorescent dyes) according to the position that will be sequenced. These molecules hybridize to the target DNA sequence, next to the anchor sequence, and DNA ligase preferentially joins the molecule to the anchor when its bases match the unknown DNA sequence. Based on the fluorescence produced by the molecule, one can infer the identity of the nucleotide at this position in the unknown sequence. The oligonucleotide probes may also be constructed with cleavable linkages which can be cleaved after identifying the label. This will both remove the label and regenerate a 5′ phosphate on the end of the ligated probe, preparing the system for another round of ligation. This cycle can be repeated several times to read longer sequences.

Nanopore sequencing is a third generation sequencing approach that works by monitoring changes to an electrical current as nucleic acids pass through a protein nanopore on a solid-state membrane. The biological or solid-state membrane, where the nanopore is found, is surrounded by electrolyte solution. The membrane splits the solution into two chambers. A bias voltage is applied across the membrane inducing an electric field that drives charged particles, in this case the ions, into motion. This effect is known as electrophoresis. For high enough concentrations, the electrolyte solution is well distributed and all the voltage drop concentrates near and inside the nanopore. This means charged particles in the solution only feel a force from the electric field when they are near the pore region. This region is often referred as the capture region. Inside the capture region, ions have a directed motion that can be recorded as a steady ionic current by placing electrodes near the membrane. Imagine now a nano-sized polymer such as a nucleic acid placed in one of the chambers. The nucleic acid also has a net charge that feels a force from the electric field when it is found in the capture region. The nucleic acid approaches this capture region aided by brownian motion and any attraction it might have to the surface of the membrane. Once inside the nanopore, the nucleic acid translocates, nucleotide-by-nucleotide, through the nanopore via a combination of electro-phoretic, electro-osmotic and sometimes thermo-phoretic forces. Inside the pore the nucleotides of the nucleic acid occupies a volume that partially restricts the flow of ions, observed as an ionic current drop. Based on various factors of the nucleotides, such as geometry, size and chemical composition, the change in magnitude of the ionic current and the duration of the translocation will vary. Different nucleotides of the nucleic acid can then be sensed and potentially identified based on this modulation in ionic current.

### Large-Scale Genomic Breakpoints

In some embodiments, the chromosome instability in the tumor sample genome is detected by measuring the number of large-scale rearrangements or large-scale genomic alteration breakpoints (LGBs) per genome in the tumor sample. In some embodiments, the LGB refers to a breakpoint between two adjacent genomic segments of different copy number, each such genomic segment being at least 1, 2, 3, 4, 5, 6, 7, 8, 9, or 10 megabases long.

A normal diploid genome has precisely two copies of each chromosome. However, when the genome of a cell becomes unstable, copy number alteration (CNA) occurs, and the number of copies varies across the genome. To determine the CNA in a genome, the genome is partitioned into maximum-length contiguous regions such that the copy number at each site within a region is the same. Each such region is called a segment, and the copy number shared by all the sites within that segment is referred to as the absolute copy number. In the case of a diploid genome with no CNAs, there is only one segment, which is the whole genome, and the absolute copy number is 2. A cell having HRD, on the other hand, has CNAs and multiple segments, in particular, segments of large size. As a result, the number of LGBs can be used as a biomarker for HRD in a cell.

In some embodiments, LGBs can be detected by a genome copy number profile generated from the WGS data. In some embodiments, the genome copy number profile is a profile of the copy number across the whole genome and comprises a parameter selected from genomic segments along the genome, copy number of the genomic segments, copy number alteration (CNA) breaks, number of CNA breaks, and a combination thereof.

In some embodiments, the genome copy profile can be generated following the following steps. In the first step, the WGS data, i.e., nucleic acid sequence reads, are mapped to a reference genome. As used herein, the term “mapping” or “mapping to a reference genome” means aligning nucleic acid sequence reads to a reference genome whose sequence is already known. Various programs and algorithms have been developed to map nucleic acid sequence reads to a reference (see, Flicek P, Birney E. (2009) Sense from sequence reads: methods for alignment and assembly, Nat Methods 6(11 Suppl): S6-S12; Neilsen R, Paul JS et al. (2011) Genotype and SNP calling from next-generation sequencing data. Nat Rev Genet 12: 443-52; Ruffalo M et al. (2011) Comparative analysis of algorithms for next-generation sequencing read alignment. Bioinformatics 27: 2790-96; Patnaik S et al. (2012) Customisation of the exome data analysis pipeline using a combinatorial approach. PLoS ONE 7: e30080). Among the various programs and algorithms, Burrows-Wheeler Aligner (BWA), which is based on Burrows-Wheeler transformation, (Li H, Durbin R (2009) Fast and accurate short rad alignment with Burrows-Wheeler transform. Bioinformatics 25: 1754-60) demonstrates a good balance between running time, memory usage and accuracy, and commonly used in different computation pipelines.

In the next step, the number of aligned WGS reads is counted in each site or region along the genome. In some embodiments, to count the number of aligned WGS reads, the genome is partitioned into fixed- or variable-sized segments or windows, which are called bins, and the number of reads within a bin is aggregated, so that the resolution at which the genome copy numbers are called is defined by bins rather than by individual sites. This is to reduce the effects of variable amplification and sequence sampling. In some embodiments, the number of aligned WGS reads is counted using bins of a fixed size (all bins have the same size), e.g., of 30, 40, 50, 60, 70, 80, 90, or 100 kilobases, preferably 50 kilobases. In some embodiments, the number of aligned WGS reads is counted using variable-size bins to avoid false-positive deletion calls (i.e., false calling of breakpoints) in repetitive regions, as a result of the removal of low mapping quality reads.

In some embodiments, the read counts are adjusted by GC correction and mappability correction. The GC correction adjusts read counts according to the GC content in the corresponding genomic region to remove the GC content’s effect on the read counts. The mappability correction adjusts read counts according to how mappable a genomic region is. The higher the repetitiveness in a region, the lower its mappability, and the lower the number of uniquely aligned reads.

The next step of the genome copy profile generation is segmentation, which identify the boundaries (physical locations) between genomic regions that have different absolute copy numbers. There are generally three approaches for segmentation: a sliding-window approach, an objective function-based approach, and an HMM-based approach.

The sliding-window approach segments the genome by statistical testing, looking for regions whose read counts differ greatly from those of the other regions. Methods that use the sliding-window approach do not calculate the absolute copy number simultaneously with segmentation. A post-processing approach is needed, which usually involves testing different candidates of ploidy and selecting the one whose resulting copy number profile is as close to integer numbers as possible.

The objective function-based approach combines the approximation to the data and the limitation of the breakpoints in one formula. Such approaches model the (normalized) read count by a piecewise constant function, so that the function (i) is in fidelity to the data as much as possible and (ii) has as few changes as possible. Like the sliding-window approach, methods based on the objective function approach do not necessarily simultaneously assign absolute copy numbers to each segment either.

In the HMM-based approach, states correspond to the different possible absolute copy numbers, and transitions between states capture the segmentation (i.e., transition out of a state at bin i denotes that bins i-1 and i belong to two different segments). Due to its ability to model hundreds of cells in one objective function, the objective function approach is more suitable for simultaneous breakpoint identification across sampled cells.

After the segmentation, the number of LGBs per genome, i.e., the number of breakpoints between two adjacent genomic segments of different copy number, in the tumor sample can then be counted. In some embodiments, only genomic segments of at least 5 megabases (preferably 6, 7, 8, 9, or 10 megabases) are counted so as to make sure that the copy number variation is caused by chromosome rearrangement. In some embodiments, all genomic segments are counted or use loose criteria (1, 2, 3, 4 megabase).

In some embodiments, the number of LGBs per genome in the tumor sample is compared to a threshold number to determine whether the patient has HRD. In some embodiments, the threshold number of LGBs is 25 per genome, i.e., it can be determined that the patient has a risk of HRD when the number of LGBs per genome in the tumor sample is greater than 25. In some embodiments, it can be determined that the risk of HRD in the patient is high when the number of LGBs is greater than 35. In some embodiments, it can be determined that the risk of HRD in the patient is moderate when the number of LGBs is greater than 25 and less than or equal to 35.

### Deep Learning Model

In some embodiments, the method disclosed herein involves using a deep learning model to determine the risk of having HRD in the patient.

Deep learning is a machine learning method based on artificial neural networks with representation learning. Artificial neural networks (ANNs) are computing systems inspired by the biological neural networks that constitute animal brains. An ANN is based on a collection of connected units called artificial neurons, (analogous to biological neurons in a biological brain). Each connection (synapse) between neurons can transmit a signal to another neuron. The receiving (postsynaptic) neuron can process the signal(s) and then signal downstream neurons connected to it. Neurons may have states, generally represented by real numbers, typically between -1 and 1. Neurons and synapses may also have a weight that varies as learning proceeds, which can increase or decrease the strength of the signal that it sends downstream. Typically, neurons are organized in layers. Different layers may perform different kinds of transformations on their inputs. Signals travel from the first (input), to the last (output) layer, possibly after traversing the layers multiple times. Deep learning refers to the use of multiple layers in the network to progressively extract higher level features from the raw input.

In some embodiments, the method disclosed herein involves using a deep learning model to predict the HRD status using CNV segment information. In some embodiments, the deep learning model comprises one or multi-layer long short-term memory (LSTM) or convolutional neural network (CNN) followed by a fully connected network. LSTM is an artificial recurrent neural network architecture, which, unlike standard feedforward neural networks, has feedback connections. CNN is an artificial neural network architecture, which uses shared-weight convolution kernels to extract features by sliding along the inputs. In some embodiments, the deep learning model generates from the WGS data a chromosome instability (CI) score for each of the 22 chromosomes.

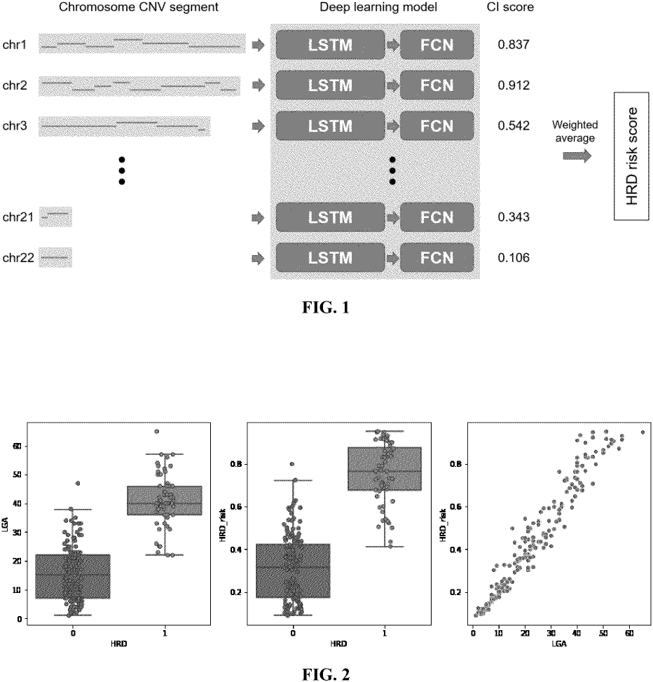

In the next step, a HRD risk score is computed by calculating an average of CI scores. In some embodiments, the HRD risk score is a weighted average of CI scores based on chromosome base length, position of each chromosome or position of each CNA break. The structure of an exemplary deep learning model used herein is illustrated in FIG. 1.

In some embodiments, to train the deep learning model, each chromosome in the sample is assigned a chromosome instability (CI) score based on the sample’s HRD label. HRD positive samples had CI score 1 for all the 22 chromosomes, while negative samples had 0. In some embodiments, the deep learning model is trained on chromosome level data to predict CI scores.

In some embodiments, the trained model is applied to the sample CNV segmentation results generated from WGS data to predict the HRD risk score. In some embodiments, the HRD risk score is a value between 0 and 1. In some embodiments, the risk of having HRD in a patient is high when the risk score > 0.6. In some embodiments, the risk of having HRD in a patient is moderate when the risk score is greater than 0.4 and less than or equal to 0.6. In some embodiments, the risk of having HRD in a patient is low when the risk score is no greater than 0.4.

### Computer-Implemented Methods, Systems and Devices

Any of the methods described herein may be totally or partially performed with a computer system including one or more processors, which can be configured to perform the steps. Thus, embodiments are directed to computer systems configured to perform the steps of any of the methods described herein, potentially with different components performing a respective step or a respective group of steps. Although presented as numbered steps, steps of methods herein can be performed at the same time or in a different order. Additionally, portions of these steps may be used with portions of other steps from other methods. Also, all or portions of a step may be optional. Any of the steps of any of the methods can be performed with modules, circuits, or other means for performing these steps.

Any of the computer systems mentioned herein may utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. The subsystems can be interconnected via a system bus. Additional subsystems include, for example, a printer, keyboard, storage device(s), monitor, which is coupled to display adapter, and others. Peripherals and input/output (I/O) devices, which couple to I/O controller, can be connected to the computer system by any number of means known in the art, such as serial port. For example, serial port or external interface (e.g. Ethernet, Wi-Fi, etc.) can be used to connect computer system to a wide area network such as the Internet, a mouse input device, or a scanner. The interconnection via system bus allows the central processor to communicate with each subsystem and to control the execution of instructions from system memory or the storage device(s) (e.g., a fixed disk, such as a hard drive or optical disk), as well as the exchange of information between subsystems. The system memory and/or the storage device(s) may embody a computer readable medium. Any of the data mentioned herein can be output from one component to another component and can be output to the user.

A computer system can include a plurality of the same components or subsystems, e.g., connected together by an external interface or by an internal interface. In some embodiments, computer systems, subsystem, or apparatuses can communicate over a network. In such instances, one computer can be considered a client and another computer a server, where each can be part of the same computer system. A client and a server can each include multiple systems, subsystems, or components.

It should be understood that any of the embodiments of the present disclosure can be implemented in the form of control logic using hardware (e.g., an application specific integrated circuit or field programmable gate array) and/or using computer software with a generally programmable processor in a modular or integrated manner. As used herein, a processor includes a multi-core processor on the same integrated chip, or multiple processing units on a single circuit board or network. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present invention may be created using a data signal encoded with such programs. Computer readable media encoded with the program code may be packaged with a compatible device or provided separately from other devices (e.g., via Internet download). Any such computer readable medium may reside on or within a single computer product (e.g. a hard drive, a CD, or an entire computer system), and may be present on or within different computer products within a system or network. A computer system may include a monitor, printer, or other suitable display for providing any of the results mentioned herein to a user.

### Methods for Treating Cancer

The method of identifying patients with HRD as described above has therapeutic applications. It is believed that a treatment of drugs which causes double strand breaks in the DNA (such as alkylating agents) or a treatment which inhibits the alternative DNA repair pathway (such as PARPi) will be more efficient if the tumor is deficient for the HR pathway. Therefore, in another aspect, the present disclosure provides a method for treating cancer in a patient having deficiency in the HR pathway. In some embodiments, the method comprises administering to the patient a therapeutically effective amount of a drug that inhibits the alternative DNA repair pathway, such as a PARP inhibitor or a drug that causes double strand breaks in the DNA, such as an alkylating agent, wherein the patient has been determined to have HRD by the method described above.

As used herein, “PARP inhibitor” refers to a compound which is capable of inhibiting the activity of the enzyme polyADP ribose polymerase (PARP), a protein that is important for repairing single-strand breaks (‘nicks’ in the DNA). If such nicks persist unrepaired until DNA is replicated (which must precede cell division), then the replication itself will cause double strand breaks to form. Drugs that inhibit PARP cause multiple double strand breaks to form in this way, and in tumors with HRD these double strand breaks cannot be efficiently repaired, leading to the death of the cells.

In some embodiments, the PARP inhibitor according to the invention can be selected from the group consisting of iniparib, olaparib, rucaparib, CEP 9722, MK 4827, BMN-673, and 3-aminobenzamide.

As used herein, the term “alkylating agent” or “alkylating antineoplastic agent” refers to compounds which attach an alkyl group to DNA. In some embodiments, the alkylating agent according to the invention can be selected from platinum complexes such as cisplatin, carboplatin and oxaliplatin, chlormethine, chlorambucil, melphalan, cyclophosphamide, ifosfamide, estramustine, carmustine, lomustine, fotemustine, streptozocin, busulfan, pipobroman, procarbazine, dacarabazine, thiotepa and temozolomide.

The drug described herein may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the drug may be administered in conjunction with other treatments.

A suitable, non-limiting example of a dosage of the drug is from about 1 mg/kg to about 2400 mg/kg per day, such as from about 1 mg/kg to about 1200 mg/kg per day, 75 mg/kg per day to about 300 mg/kg per day, including from about 1 mg/kg to about 100 mg/kg per day. Other representative dosages of such agents include about 1 mg/kg, 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, 250 mg/kg, 300 mg/kg, 400 mg/kg, 500 mg/kg, 600 mg/kg, 700 mg/kg, 800 mg/kg, 900 mg/kg, 1000 mg/kg, 1100 mg/kg, 1200 mg/kg, 1300 mg/kg, 1400 mg/kg, 1500 mg/kg, 1600 mg/kg, 1700 mg/kg, 1800 mg/kg, 1900 mg/kg, 2000 mg/kg, 2100 mg/kg, 2200 mg/kg, and 2300 mg/kg per day. In some embodiments, the dosage of the drug in human is about 400 mg/day given every 12 hours. In some embodiments, the dosage of the drug in human ranges 300-500 mg/day, 100-600 mg/day or 25-1000 mg/day. The effective dose of drug disclosed herein may be administered as two, three, four, five, six or more sub-doses, administered separately at appropriate intervals throughout the day.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### Example 1

### Materials and Methods

### Extreme Low Coverage Whole Genome Sequencing (WGS) of Formalin-Fixed Paraffin-Embedded (FFPE) Tumor Tissue Samples

Tissue sample processing and database building process:

To prepare the DNA library for sequencing from fresh surgical tissue or FFPE samples, DNA extraction was performed on the obtained patient samples using the GeneRead DNA FFPE Kit (Qiagene). The extracted DNA was tested for concentration and purity. 200 ng of extracted DNA per sample was used as the input material for library preparation. The NEBNext® Ultra™ II library preparation kit was used to generate the NGS sequencing library, and the index code was added during the library preparation process into each sample. After purification of the PCR products, the size distribution of the libraries was analyzed and quantified by real-time PCR. Libraries were sequenced on the Illumina NovaSeq platform.

To prepare the DNA library for sequencing from CTC blood sample, commercial platforms (including but not limited to CTC biological properties and separation methods based on their physical properties) were used on the obtained patient samples (fresh blood) to enrich and isolate CTCs (circulating tumor cells). The Single-Cell Whole Genome Amplification Kit was used to perform CTC whole-genome amplification. After the concentration of the amplified whole genome DNA was determined, 200 ng of DNA was used as the input material for library preparation for each sample, and the NEBNext® Ultra™ II library preparation kit was used to generate the NGS sequencing library. The index code was added to each sample for index during the library preparation process. After purification of the PCR products, the size distribution of the libraries was analyzed and quantified by real-time PCR. Libraries were sequenced on an Illumina sequencing platform.

### Genome Copy Number Profiling

The original sequencing data was aligned to the reference genome using BWA with QC filtering and duplicate removal. Unique aligned reads with good mapping quality were then counted along the genome using a 50,000 bp long sliding window. Read counts were adjusted with genomic GC content and mappability for each window. HMMcopy was called on the window read counts in its default setting to obtain the genome copy number profile of human chromosome 1 to 22 (chromosome X and Y were ignored in the analysis).

### Large Scale Genomic Breakpoints (LGBs) Determination

Large scale genomic breakpoints (LGBs) were defined and calculated through the following steps: (i) Genomic segments shorter than 3Mb were removed; (ii) For each chromosome, copy number alteration (CNA) breaks were counted when the two adjacent segments are both longer than 10 Mb; and (iii) The number of CNA breaks was summed for all 22 chromosomes to get the final LGB score. The HRD high risk group was defined as LGB > 35, middle risk group as 25 < LGB <= 35, low risk group as LGB <= 25.

### Predict HRD Risk Score by HRD Risk Test

The inventors developed HRD risk test, a deep learning based model to predict sample HRD status using CNV segment information. HRD risk test consists of two components: (i) the first component is a 1-layer LSTM followed by a fully connected network, which generates a chromosome instability (CI) score for each of the 22 chromosomes; (ii) the second component is to compute a HRD risk score by calculating a weighted average (chromosome base length as weight) of CI scores. The whole model structure is illustrated in FIG. 1.

To train the deep learning model, each chromosome in the sample was assigned a chromosome instability (CI) score based on the sample’s HRD label. HRD positive samples had CI score 1 for all the 22 chromosomes, while negative samples had 0. The deep learning model was trained on chromosome level data to predict CI scores. Model was trained on 80 samples (28 HRD positive) with 1760 chromosomes and validated on 93 samples (27 HRD positive) with 2046 chromosomes.

The trained model was applied to the sample CNV segmentation results generated by HMMcopy to predict the HRD risk score. HRD risk score is a value between 0 and 1. The HRD high risk group is defined as risk score > 0.6, middle risk group as 0.4 < risk score <= 0.6, low risk group as risk score <= 0.4.

### Example 2

This example illustrates the consistency measurement of shallow sequencing approaches (LGB and HRD risk score) with a deep sequencing method.

Deep WGS data of 191 breast cancer patient samples were downloaded from the European Genome-Phenome Archive (EGA) (Nik-Zainal, S. et al. (2016) Landscape of somatic mutations in 560 breast cancer whole-genome sequences. Nature, 534(7605), 47-54). The HRD label of the patient data was obtained from HRDetect (Davies, H. et al. (2017) HRDetect is a predictor of BRCA1 and BRCA2 deficiency based on mutational signatures. Nature medicine, 23(4), 517-525). The original WGS data was down-sampled to 0.5 genome coverage to simulate the ultra low coverage WGS. The HDR risk method was then applied to calculate the LGB score. HRD risk test was trained on 80 samples and validated on 111 samples as described in Example 1. The performance of the HDR risk method was evaluated using the HRDetect predicted label as ground truth. FIG. 2 shows that both LGB and HRD risk score were able to well separate the HRD negative and positive groups with a high AUROC equals 0.968 and 0.974 respectively. For HRD risk test, the AUROC of training data is 0.990, and 0.962 for validation data. The consistency of the two approaches were also checked. A strong linear correlation was found between the LGB score and HRD risk score by HRD risk test.

### Example 3

This example illustrates the robustness of the method disclosed herein in data of extreme low sequencing depth.

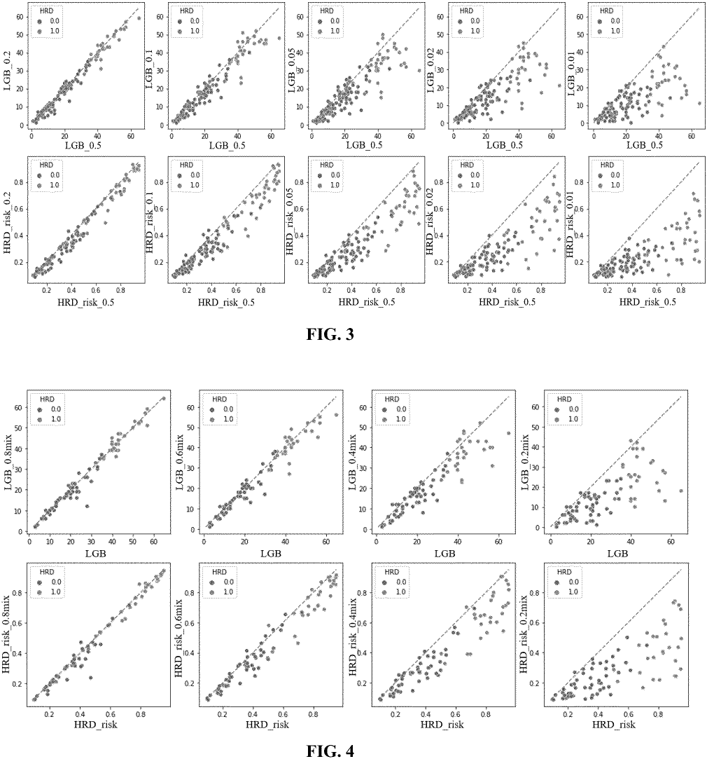

The 130 WGS samples were further down-sampled to even lower coverage of 0.2, 0.1, 0.05, 0.02 and 0.01 to examine the detection limit of sequencing depth of the methods disclosed herein. Good correlations between the LGB scores calculated in 0.5 coverage sample and those calculated in 0.2, 0.1, and 0.05 coverage were observed (Table 1, FIG. 3). Although a general consistency still existed when the coverage was below 0.05, the AUROC started to fall behind the optimal range. Overall, the method disclosed herein is capable of identifying HRD in extreme low coverage WGS data whose depth is as low as 0.05.

TABLE 1

AUROCs of LGB and HRD risk scores of different coverage

| | | | | | | |
|---|---|---|---|---|---|---|
| Depths | 0.5 | 0.2 | 0.1 | 0.05 | 0.02 | 0.01 |
| AUROC of LGB | 0.976 | 0.978 | 0.975 | 0.960 | 0.941 | 0.919 |
| AUROC of HRD risk score | 0.982 | 0.980 | 0.977 | 0.966 | 0.940 | 0.909 |

### Example 4

This example illustrates the robustness of the method disclosed herein when applied to the samples with low tumor fraction.

A common observation of clinical tumor samples is that the tumor fractions in FFPE samples vary, and low tumor fractions can potentially harm the sensitivity of analysis algorithms. To examine the robustness of the method disclosed herein to low tumor proportion, the inventors generated admixture samples with 80%, 60%, 40% and 20% of tumor proportion by randomly sampling the reads from the original tumor samples and the matched normal samples with the corresponding probabilities. Same analysis was carried out to calculate the LGB and HRD risk scores. The inventors observed good correlations between the LGB and HRD risk scores from the original samples and diluted samples (FIG. 4). Even when the simulated samples contained 60% less tumor DNA, the sensitivity and specificity of our method were only minimally affected.

To get a sense of the actual detection limit of tumor fraction of the method disclosed herein, the inventors ran Sequenza (Favero, F. et al. (2015) Sequenza: allele-specific copy number and mutation profiles from tumor sequencing data. Annals of Oncology, 26(1), 64-70) to estimate the tumor purity in the original samples. The purities of diluted samples were calculated by multiplication of the estimated purity in the original samples and the corresponding dilution coefficients. The original samples and the diluted samples of different degrees were then combined together to form the final analysis pool, and the AUROCs for each purity group were calculated. The inventors found that our method was able to achieve a good detection performance of HRD in low tumor fraction.

### Example 5

This example further validates the robustness of the LGB score as a biomarker for HRD by comparing with the conventional method of BRCA analysis test.

BRCA analysis test was conducted on a cohort of 75 breast cancer patients (in Harbin, China), 25 of which were BRCA positive, and the rest 50 were BRCA negative. As shown in Example 4 and FIG. 4, our method maintained good sensitivity and specificity even when the tumor proportion of the sample is as low as 20%.

BRCA test: BRCA analysis test was performed using a BRCA mutation test kit based on next generation sequencing (NGS).

Briefly, total DNA were used for DNA sequencing library prep, and genomic region of BRCA1 and BRCA2 were enriched using target probes. The enriched library was used for sequencing, and then the mutation status of BRCA½ was analyzed based on sequencing results.

Based such finding, 42 samples (9 BRCA positive and 33 BRCA negative) with tumor proportion greater than 20% were prepared and used for the validation of the methods disclosed herein. For each specimen, the tumor proportion was assessed using FFPE sections by pathology review.

As shown in FIG. 5, at various sequencing depth from 0.01 to 0.5, through the BRCA-positive LGB test, the result shows LGB score was significantly higher than that of BRCA-negative samples, indicating that the LGB score is able to distinguish between BRCA-positive and BRCA negative samples.

As shown in Table 2, the data filled in the “Deficient” column and “Intact” column mean the number of samples of each classification with the label of the sequencing depth and LGB score; the data filled in the “BRCA positive rate” column mean the proportion of BRCA positive samples to the total 42 samples; the data filled in the “LGB positive rate” column mean the proportion of LGB positive samples to the total 42 samples.

TABLE 2

HRD results of LGB test

| | LGB score | A(0.5X) | B(0.2X) | C(0.1X) | D(0.05X) |
|---|---|---|---|---|---|
| Deficient (BRCA positive) | >=30 | 7 | 6 | 6 | 5 |
| | <30 | 2 | 3 | 3 | 4 |
| Intact (BRCA negative) | >=30 | 16 | 11 | 10 | 9 |
| | <30 | 17 | 22 | 23 | 24 |
| BRCA positive rate | | 21.4% | 21.4% | 21.4% | 21.4% |
| LGB positive rate | | 54.8% | 40.5% | 38.1% | 33.3% |

Furthermore, the detection rate of HRD positive sample by LGB score was much greater than that measured by conventional BRCA analysis method (i.e., BRCA positive rate) under the conditions of 0.5, 0.2, 0.1 and 0.05 sequencing depth (Table. 2), indicating that the application scope of LGB score for HRD detection is much broader than that of the conventional BRCA analysis method.

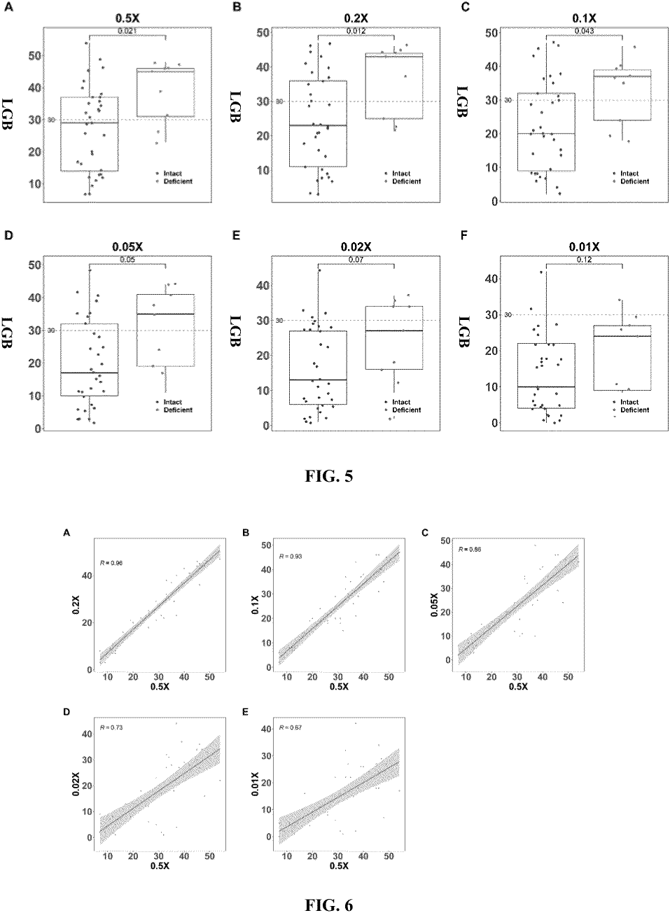

Also, the robustness of the LGB method was illustrated by the good correlations between the LGB score detected with 0.5 sequencing depth and that detected with lower sequencing depth at 0.01 and 0.2 (FIG. 6). Particularly, the R values show decent correlation when the coverage was 0.1 or 0.2, indicating that optimal detection results of the LGB method could be guaranteed even with a sequencing depth as low as 0.1.

### Example 6

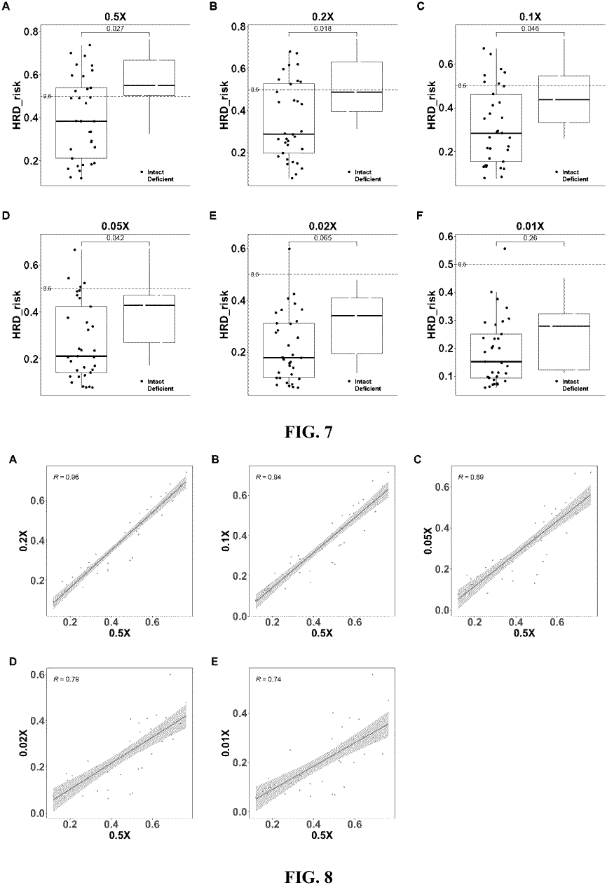

This example further validates the robustness of the HRD-risk score as a biomarker for HRD by comparing with the conventional method of BRCA analysis test. Similar to EXAMPLE 5, the HRD risk testing was applied to the 42 samples. As shown in FIG. 7, at various sequencing depth from 0.01 to 0.5, the BRCA-positive HRD risk score was significantly higher than that of BRCA-negative samples, indicating that the HRD risk score is able to well distinguish between BRCA-positive and BRCA negative samples. As shown in Table 3, the data filled in the “Deficient” column and “Intact” column mean the number of samples of each classification with the label of the sequencing depth and HRD risk score; the data filled in the “BRCA positive rate” column mean the proportion of BRCA positive samples to the total 42 samples; the data filled in the “HRD risk positive rate” column mean the proportion of HRD risk positive samples to the total 42 samples.

TABLE 3

HRD results of HRD risk test

| | HRD risk score | A(0.5X) | B(0.2X) | C(0.1X) | D(0.05X) | E(0.02X) | F(0.01X) |
|---|---|---|---|---|---|---|---|
| Deficient (BRCA positive) | >=0.5 | 7 | 4 | 4 | 2 | 0 | 0 |
| | <0.5 | 2 | 5 | 5 | 7 | 9 | 9 |
| Intact (BRCA negative) | >=0.5 | 11 | 9 | 7 | 4 | 1 | 1 |
| | <0.5 | 22 | 24 | 26 | 29 | 32 | 32 |
| BRCA positive rate | | 21.4% | 21.4% | 21.4% | 21.4% | 21.4% | 21.4% |
| HRD risk positive rate | | 42.9% | 31.0% | 26.2% | 14.3% | 2.4% | 2.4% |

Furthermore, the robustness of the HRD risk testing was illustrated by the good correlations between the HRD risk score detected with 0.5 sequencing depth and that detected with lower sequencing depth from 0.01 to 0.2 (FIG. 8). Particularly, the R values show decent correlation when the coverage was 0.05, 0.1 or 0.2, indicating that optimal detection results of the LGB method could be guaranteed even with a sequencing depth as low as 0.05.

According to the results of HRD risk testing, the best cutoff values fluctuate for different sequencing depths. The best cut-off value for 0.2 and 0.1 sequencing depths are both 0.3 while the best cut-off value for 0.05 or 0.02 sequencing depth is 0.25 or 0.2, respectively. Also, the analytical results of the HRD risk testing at these cutoff values were shown in Tables 4, 5 and 6. (Note: for a sequencing depth of 0.5, the optimal cutoff value is 0.5).

TABLE 4

Results of HRD risk test with 0.1 and 0.2 sequencing depths

| | HRD risk score | B(0.2X) | C(0.1X) |
|---|---|---|---|
| Deficient (BRCA positive) | >=0.3 | 9 | 8 |
| | <0.3 | 0 | 1 |
| Intact (BRCA negative) | >=0.3 | 14 | 14 |
| | <0.3 | 19 | 19 |
| BRCA positive rate | -- | 21.4% | 21.4% |
| HRD risk positive rate | -- | 54.8% | 52.4% |

TABLE 5

Results of HRD risk test with 0.05 sequencing depth

| | HRD risk score | D(0.05X) |
|---|---|---|
| Deficient (BRCA positive) | >=0.25 | 8 |
| | <0.25 | 1 |
| Intact (BRCA negative) | >=0.25 | 13 |
| | <0.25 | 20 |
| BRCA positive rate | -- | 21.4% |
| HRD risk positive rate | -- | 50% |

TABLE 6

Results of HRD risk test with 0.02 sequencing depth

| | HRD risk score | E(0.02X) |
|---|---|---|
| Deficient (BRCA positive) | >=0.20 | 7 |
| | <0.20 | 2 |
| Intact (BRCA negative) | >=0.20 | 14 |
| | <0.20 | 19 |
| BRCA positive | -- | 21.4% |
| HRD risk positive rate | -- | 50% |

### Example 7

This example compares the LGB score assay and the HRD risk score assay with the commercially available Myriad MyChoice® CDx test (Myriad Genetics, Salt Lake City, UT, USA) .

Myriad MyChoice® CDx test was applied to three samples in comparison with the results detected by the methods described herein. The results of the Myriad test, LGB test and HRD risk test and their corresponding scores under the conditions of various sequencing depths were summarized in Tables 7, 8 and 9.

TABLE 7

HRD results detected with 0.5 sequencing depth

| Sample ID | LGB Score | LGB Test Result | HRD Risk Score | HRD risk Test Result | Myriad HRD Score | Myriad HRD Test Result |
|---|---|---|---|---|---|---|
| JYHEB_15 | 26 | Negative | 0.444 | Negative | 33 | Negative |
| JYHEB_7 | 23 | Negative | 0.326 | Negative | 30 | Negative |
| JYHEB_19 | 31 | Positive | 0.503 | Positive | 53 | Positive |

Note: 1. Myriad threshold score ≥ 42 is positive, otherwise negative.
2. LGB threshold score ≥ 30 is positive, otherwise negative.
3. HRD risk threshold score ≥ 0.5 is positive, otherwise negative.

TABLE 8

HRD results detected with 0.2 sequencing depth

| Sample ID | LGB Score | LGB Test Result | HRD Risk Score | HRD risk Test Result | Myriad HRD Score | Myriad HRD Test Result |
|---|---|---|---|---|---|---|
| JYHEB_15 | 25 | Negative | 0.292 | Negative | 33 | Negative |
| JYHEB_7 | 22 | Negative | 0.245 | Negative | 30 | Negative |
| JYHEB_19 | 30 | Positive | 0.501 | Positive | 53 | Positive |

Note: 1. LGB threshold score ≥ 30 is positive, otherwise negative.
2. HRD risk threshold score ≥ 0.3 is positive, otherwise negative.

TABLE 9

HRD results detected with 0.1 sequencing depth

| Sample ID | LGB Score | LGB Test Result | HRD Risk Score | HRD risk Test Result | Myriad HRD Score | Myriad HRD Test Result |
|---|---|---|---|---|---|---|
| JYHEB_15 | 19 | Negative | 0.166 | Negative | 33 | Negative |
| JYHEB_7 | 24 | Negative | 0.201 | Negative | 30 | Negative |
| JYHEB_19 | 30 | Positive | 0.490 | Positive | 53 | Positive |

Note: 1. LGB threshold score ≥ 30 is positive, otherwise negative.
2. HRD risk threshold score ≥ 0.3 is positive, otherwise negative.

The results of the LGB test and HRD risk test of the present disclosure were accord to that of the Myriad method under all conditions of different sequencing depth, which confirming that both methods described herein are able to detect the HRD in cancer patients.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

### REFERENCE

Lord, C. J., & Ashworth, A. (2012) The DNA damage response and cancer therapy. Nature, 481(7381), 287-294.

Venkitaraman, A. R. (2014) Cancer suppression by the chromosome custodians, BRCA1 and BRCA2. Science, 343(6178), 1470-1475.

Nik-Zainal, S., Davies, H., Staaf, J., Ramakrishna, M., Glodzik, D., Zou, X., ... & Stratton, M. R. (2016). Landscape of somatic mutations in 560 breast cancer whole-genome sequences. Nature, 534(7605), 47-54.

Coleman, R. L. et al. (2019) Veliparib with first-line chemotherapy and as maintenance therapy in ovarian cancer. New England Journal of Medicine, 381(25), 2403-2415.

Davies, H. et al. (2017) HRDetect is a predictor of BRCA1 and BRCA2 deficiency based on mutational signatures. Nature medicine, 23(4), 517-525.

Favero, F. et al. (2015) Sequenza: allele-specific copy number and mutation profiles from tumor sequencing data. Annals of Oncology, 26(1), 64-70.

What is claimed is:

1. A method for detecting deficiency in the DNA homologous recombination pathway in a patient having cancer, the method comprising:
obtaining a whole genome sequencing (WGS) data of a tumor sample from the patient;
generating from the WGS data a genomic alteration profile including genome copy number profile, fusion profile, translocation profile, or inversion profile;
generating from the genome copy number profile a chromosome instability (CI) scores for each chromosome using a deep learning model, thus generating a set of CI scores;
generating from the set of CI scores a risk score of deficiency in the DNA homologous recombination pathway;
determining that the risk score is greater than a threshold number; and
identified the patient as having a risk of deficiency in the DNA homologous recombination pathway.

2. The method of claim 1, wherein (i) the threshold number is 0.4; or (ii) the risk of deficiency in the DNA homologous recombination pathway is high when the risk score is greater than 0.6; or (iii) the risk of deficiency in the DNA homologous recombination pathway is moderate when the risk score is greater than 0.4 and less than or equal to 0.6.

3. The method of claim 1, wherein the tumor sample is an FFPE sample or a CTC sample.

4. The method of claim 1, wherein the cancer is selected from breast cancer, ovary cancer, pancreas cancer, head and neck carcinoma and melanoma.

5. The method of claim 1, wherein the genome copy number profile comprises a parameter selected from genomic segments along the genome, copy number of the genomic segments, copy number alteration (CNA) breaks, number of CNA breaks, and a combination thereof.

6. The method of claim 1, wherein the genome copy number profile is generated by:
aligning the WGS data to a reference genome, thereby generating a group of aligned WGS reads along the genome,
counting the group of aligned WGS reads along the genome, and
generating genomic segments along the genome, wherein two adjacent genomic segments have significantly different numbers of read counts.

7. The method of claim 1, wherein the deep learning model comprises one or more layers of long short-term memory or convolutional neural network and a fully connected network.

8. The method of claim 1, wherein the risk score is an average of the set of CI scores weighted by base length of each chromosome.

9. A method for treating cancer in a patient, the method comprising
administering to the patient a therapeutically effective amount of a PARP inhibitor and/or an alkylating agent,
wherein the patient has been identified as having a risk of deficiency in the DNA homologous recombination pathway by the method of claim 1.

10. The method of claim 9, wherein the PARP inhibitor and/or alkylating agent is selected from iniparib, Olaparib, rucaparib, CEP9722, MK4827, BMN673, 3-aminobenzaide, platinum complexes, chlormethine, chlorambucil, melphalan, cyclophosphamide, ifosfamide, estramustine, carmustine, lomustine, fotemustine, streptozocin, busulfan, pipobroman, procarbazine, dacarbazine, thiotepa and temozolomide.

11. A non-transitory computer readable medium having instructions stored thereon, wherein the instructions, when executed by a processor, cause the processor to perform a method comprising:
obtaining a whole genome sequencing (WGS) data of a tumor sample from a patient;
generating from the WGS data a genomic alteration profile including genome copy number profile, fusion profile, translocation profile, or inversion profile;
generating from the genome copy number profile a chromosome instability (CI) scores for each chromosome using a deep learning model, thus generating a set of CI scores; and
generating from the set of CI scores a risk score of deficiency in the DNA homologous recombination pathway.

12. The non-transitory computer readable medium of claim 11, wherein the method further comprises:
determining that the risk score is greater than a threshold number; and
identified the patient as having a risk of deficiency in the DNA homologous recombination pathway.

13. A method for detecting deficiency in the DNA homologous recombination pathway in a patient having cancer, the method comprising:
obtaining a whole genome sequencing (WGS) data of a tumor sample from the patient, wherein the WGS data has a sequencing depth of 0.05 to 0.5;
generating from the WGS data a genome copy number profile;
generating from the genome copy number profile a number, per genome, of large-scale genomic breakpoints (LGBs), wherein the LGB is a breakpoint between two adjacent genomic segments of a LGB such as different copy number, each such genomic segment being at least 10 megabases long;
determining that the number of LGBs is greater than a threshold number of LGBs; and
identified the patient as having a risk of deficiency in the DNA homologous recombination pathway.

14. The method of claim 13, wherein (i) the threshold number of LGBs is 25; or (ii) the risk of deficiency in the DNA homologous recombination pathway is high when the number of LGBs is greater than 35; or (iii) the risk of deficiency in the DNA homologous recombination pathway is moderate when the number of LGBs is greater than 25 and less than or equal to 35.

15. The method of claim 13, wherein the tumor sample is an FFPE sample or a CTC sample.

16. The method of claim 13, wherein the cancer is selected from breast cancer, ovary cancer, pancreas cancer, head and neck carcinoma and melanoma.

17. The method of claim 13, wherein the genome copy number profile is generated by:
aligning the WGS data to a reference genome, thereby generating a group of aligned WGS reads along the genome,
counting the group of aligned WGS reads along the genome, and
generating genomic segments along the genome, wherein two adjacent genomic segments have significantly different numbers of read counts.

18. A method for treating cancer in a patient, the method comprising
administering to the patient a therapeutically effective amount of a PARP inhibitor and/or an alkylating agent,
wherein the patient has been identified as having a risk of deficiency in the DNA homologous recombination pathway by the method of claim 12.

19. The method of claim 18, wherein the PARP inhibitor and/or alkylating agent is selected from iniparib, Olaparib, rucaparib, CEP9722, MK4827, BMN673, 3-aminobenzaide, platinum complexes, chlormethine, chlorambucil, melphalan, cyclophosphamide, ifosfamide, estramustine, carmustine, lomustine, fotemustine, streptozocin, busulfan, pipobroman, procarbazine, dacarbazine, thiotepa and temozolomide.

20. A non-transitory computer readable medium having instructions stored thereon, wherein the instructions, when executed by a processor, cause the processor to perform the method of claim 13.
